(19) United States
(12) Patent Application Publication
(10) Pub. No.: US 2009/0048652 A1
(43) Pub. Date: Feb. 19, 2009

(54) MEDICAL DEVICE HAVING PLASMA POLYMERIZED COATING AND METHOD THEREFOR

(75) Inventors: Shamim M. Malik, Temecula, CA (US); Paul E. Zarembo, Vadnais Heights, MN (US); Larry L. Hum, Cottage Grove, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc, St. Paul, MN (US)

(21) Appl. No.: 11/838,026

(22) Filed: Aug. 13, 2007

(51) Int. Cl. A61N 1/04; B05D 5/12

(52) U.S. Cl. 607/116; 427/2.31

(57) ABSTRACT

A medical device having at least one plasma polymerized coating allowing for a first component to be coupled with a second component.

### TECHNICAL FIELD

This relates to the field of medical devices, and more specifically to a medical device having a plasma polymerized coating.

### TECHNICAL BACKGROUND

Medical devices, such as implantable leads and pulse generators, include components that are joined together at joints and, the components and their joints are subjected to a wet environment. Over long periods of time, the stability of the joint can degrade in wet environments, and joints made with adhesives also can degrade in such an environment.

### SUMMARY

A method for creating an implantable medical device includes evacuating a chamber, introducing a material into the chamber, such as methane. The method further includes ion implanting a first portion of a first component of the medical device with the material, and depositing a coating on the first portion of the first component using plasma polymerization. Optionally, depositing the coating includes depositing the coating on at least one of a metal component or a silicone rubber component.

Options for the method include, but are not limited to, bonding the first component with a second component using the plasma polymerized coating, or applying energy to the first component, such as thermal heating the first portion of the first component, or applying laser fusion to the first portion of the first component. Further options for the method include ionizing a second portion of a second component with the material, depositing a second coating on the second portion of the second component using plasma polymerization, and bonding the first portion of the first component with the second portion of the second component. In yet another option, the method includes treating the first component with at least one of oxygen or hydrogen, and functionalizing the plasma polymerized coating.

A method includes disposing a component within a chamber, evacuating the chamber and reducing pressure within the chamber, introducing a material into the chamber, generating a plasma polymerized coating using the material, and depositing the coating on a first portion of a first implantable medical component, for instance depositing the coating on a joint of the first implantable medical component, and bonding the joint with another component.

Further options for the method include bonding the first implantable medical component with a second implantable medical component using the plasma polymerized coating, or creating OH molecules on the first implantable medical component. Other options include, but are not limited to disposing adhesive on the first implantable medical component and bonding the first implantable medical component with a second implantable medical component. In yet another option, the method includes concentrating the plasma polymerized coating on portions of the first component. In a further option, the method includes depositing a second plasma polymerized coating on a second portion of a second component, and bonding the first portion of the first component with the second portion of the second component, and optionally treating the first implantable medical component and the second implantable medical component with at least one of oxygen or hydrogen, and functionalizing the plasma polymerized coating.

An implantable medical device includes a first component having a first plasma polymerized portion deposited thereon, and a second component having a second plasma polymerized portion deposited thereon, where the first plasma polymerized portion is bonded with the second plasma polymerized portion and forms a joint for the medical device. In an option, at least one of the first component or the second component includes an electronically conductive metallic component having a surface enhanced embedded layer, and/or at least one of polyurethane or silicone rubber.

Options for the device further include medical adhesive disposed between the first plasma polymerized portion and the second polymerized portion. In a further option, the first component has a surface treated portion associated with the first plasma polymerized portion, and optionally the surface treated portion includes an ionized portion. Further options for the device are the first component includes at least one of an electrode, a terminal, or a lead body.

### BRIEF DESCRIPTION OF THE DRAWINGS

FIG. 1 illustrates a system of medical devices in accordance with at least one embodiment.

FIG. 2 illustrates a joint of a medical device in accordance with at least one embodiment.

FIG. 3 illustrates a joint of a medical device in accordance with at least one embodiment.

FIG. 4 illustrates a flow chart for creating a medical device in accordance with at least one embodiment.

FIG. 5A illustrates a chamber for use in creating a medical device in accordance with at least one embodiment.

FIG. 5B illustrates a sample to be disposed within the chamber of FIG. 5A for use in creating a medical device in accordance with at least one embodiment.

FIG. 6 illustrates samples for use in a chamber for creating a medical device in accordance with at least one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

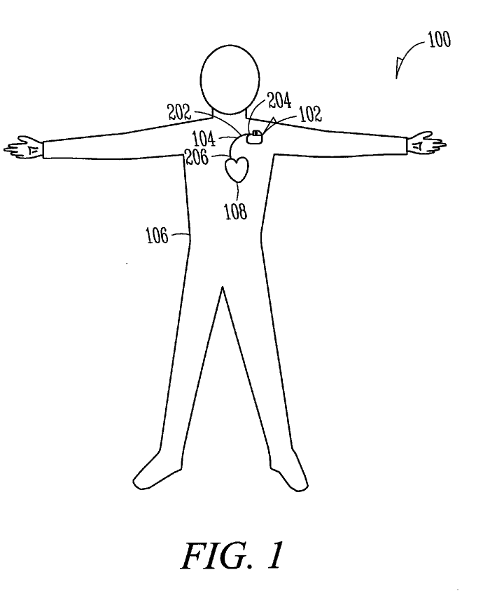

FIG. 1 illustrates a lead system 100 and an environment 106 (e.g., a subcutaneous pocket made in the wall of a subject's chest, abdomen, or elsewhere) in which the lead system 100 may be used. In varying examples, the lead system 100 may be used for delivering or receiving electrical pulses or signals to stimulate or sense a heart 108 of a patient. As shown in FIG. 1, the lead system 100 includes one or more implantable medical devices such as implantable device 102 and an implantable medical device lead 104. The implantable device 102 generically represents, but is not limited to, cardiac function management (referred to as “CFM”) systems such as pacers, cardioverters/defibrillators, pacers/defibrillators, biventricular or other multi-site resynchronization or coordination devices such as cardiac resynchronization therapy (referred to as “CRT”) devices, sensing instruments, or drug delivery systems.

Among other things, the implantable device 102 includes a source of power as well as an electronic circuitry portion. In one example, the electronic circuitry includes microprocessors to provide processing, evaluation, and to determine and deliver electrical shocks or pulses of different energy levels and timing for ventricular defibrillation, cardioversion, or pacing of heart 108 in response to sensed cardiac arrhythmia including fibrillation, tachycardia, or bradycardia. In another example, the implantable device 102 is a battery-powered device that senses intrinsic signals of the heart 108 and generates a series of timed electrical discharges.

The lead 104 includes a lead body 202 extending from a lead proximal end portion, where it is couplable with the implantable device 102. The lead 104 extends to a lead distal end portion 206, which is positioned within, on, or near tissue to be stimulated, such as a heart 108. The lead 104 further includes at least one electrode that electrically links the lead 104 with the heart 108. At least one conductor is disposed within the lead body 202 and electrically couple the at least one electrode with a terminal end of the lead 104 at the lead proximal end portion 204 and the implantable device 102.

The implantable medical devices discussed above and below include components that further include a joint, coupling a first component with a second component to form another component, for example. Examples of a first component or a second component include, but are not limited to, an electrode, a terminal, a lead body, a metal component, a polyurethane component, or a silicone rubber component. In a further option, the first component includes a polymer component, such as silicone or polyurethane, and the second component includes a metal component, such as platinum, stainless steel, MP, or titanium.

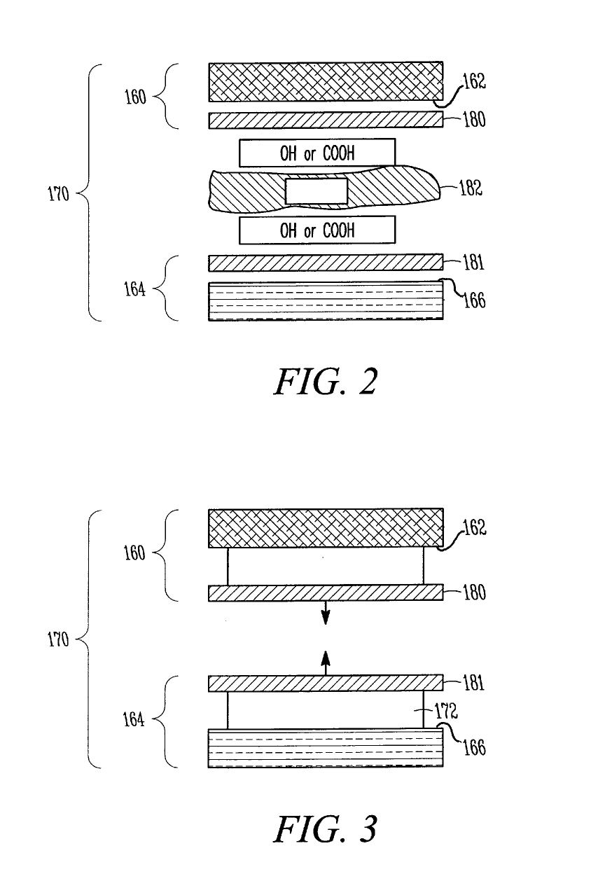

Referring to FIGS. 2 and 3, a first component 160 having a first portion 162, and a second component 164 having a second portion 166 are shown. The first component 160 and the second component 164 are brought together and are coupled together to form a joint 170.

The first component 160 includes, in an option, a first plasma polymerized portion 180 deposited thereon. For instance, a coating of plasma polymerized material is deposited on the first portion 162 using a plasma polymerizing process. For instance, a gas is introduced in to a chamber to create a plasma polymer. In an example, gases containing monomers can be created by vaporizing a liquid and introducing it into the plasma chamber, or can be introduced as a gas. Methane is one option for a material to create the plasma polymer. In an option, the second component 164 includes a second plasma polymerized portion 181 deposited thereon using a plasma polymerizing process. For instance, a coating of plasma polymerized material, such as methane, is deposited on the second portion 166. The first plasma polymerized portion 180 is bonded with the second plasma polymerized portion 181, in an option, and forms a joint for the medical device. In an option, a coating deposited using plasma polymerization is used as the interlayer for the joint of the components that are bonded together. The bonding can be achieved, in an option, using a bonding material such as adhesive 182. Other bonding materials include, but are not limited to, epoxies, UV cure adhesives, polyurethane adhesives, or a combination thereof. In another option, the bonding can occur using energy such as heat and/or pressure, where adhesive would be optional. In another option, laser bonding can be used to couple the first component 160 and the second component 164, and bonding material such as adhesive is optional.

In an option, the first portion 162 and/or the second portion 166 is treated prior to the bonding process. This can occur with or without the plasma polymerizing process. For example, the first portion 162 and/or the second portion 166 include ion implantation material, such as, but not limited to, methane. The gases used for ion implantation would include the material that is to be implanted. For implanting carbon, for example, methane can be used. At least some of the ions are buried under a surface of the component when accelerated ions are impinged on the surface with high kinetic energy. In another example, the first portion 162 and/or the second portion 166 include a surface enhanced embedded layer, such as, but not limited to, from the modified surface.

In a further option, the first portion 162 and/or the second portion 166 are surface treated with a material, such as, but not limited to, hydrogen, oxygen, or oxide plasma, or a combination thereof In another option, medical adhesive 182 is disposed between the first component 160 and the second component 164 prior to bonding the first and second components 160, 164.

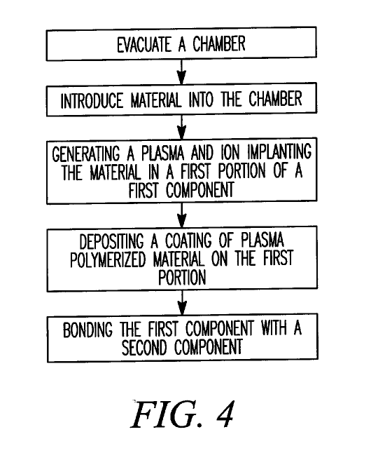

A method for forming the medical device is further provided herein. FIG. 4 illustrates a method flow chart. The method includes evacuating a chamber, introducing material into the chamber, ionizing the material in at least a first portion of a first component and/or a second portion of a second component. In a further option, the method includes depositing a coating of plasma polymerized material on the first portion of the first component. In yet another option, the method includes depositing a coating of plasma polymerized material on a second component. In a further option, the components are bonded together. In an option, the components are bonded using energy such as heat or pressure, and/or include a layer of medical adhesive between the first and second components.

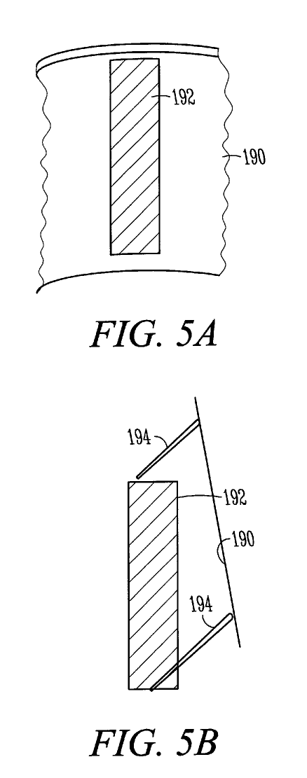

In discussing the method in further detail, the first and/or second components are optionally pre-cleaned, for example, using isopropyl. The first and/or second components are fixed within a chamber 190, for instance, as shown in FIGS. 5A and 5B. Element 192 represents an example of the first and/or second components portions of the first and/or second components, or a carrier for holding the first and/or second components within the chamber 190, each of which can have a number of shapes or sizes, and include portions of medical devices. Element 192 further includes portions of medical devices forming a joint of the medical device. The first and/or second components, portions thereof, or carriers for the first and/or second components can be secured within the chamber 190 with one or more supports 194.

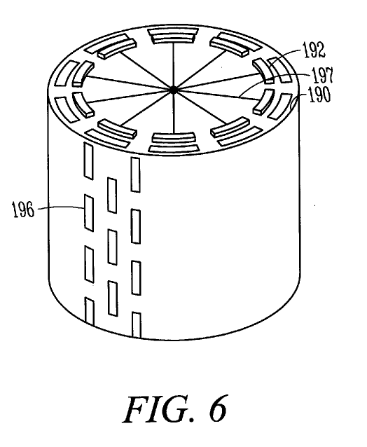

In an option, an argon plasma is used to mechanically scrub the surface of the first and/or second components. In an option, the first and/or second component include, but are not limited to a metal component, a polymer component, or a silicone rubber component, aluminum, aluminum oxide, silicon carbide, silicon oxymitride, or polyurethane tubes, PEEK, ETFE, PTFE, or combinations thereof. The metal component can include components made of titanium, stainless steel, MP35, platinum, or a combination thereof. In an option, the first and/or second component, an example of which is shown at 192, are mounted within the chamber 190 as shown in FIG. 5B or FIG. 6.

The chamber 190 is evacuated of pressure, for instance using a pump. In an option, the chamber is evacuated to a pressure of less than about 0.01 mTorr. A material, such as, but not limited to, methane is introduced into the chamber, while maintaining the pressure of less than about 0.01 mTorr. Other materials can be used including, but not limited to, ethylene, acetylene, acrylic acid, or tetra methyl silene. In an option, acrylic acid is mixed with ethylene to achieve hydrophobic coating.

In an option, the material is ionized (plasma is generated) in the first and/or second component, where the material is ion implanted into the components through a train of negative high voltage pulses applied to the components. The ion implant allows for the surface properties to be changed, while allowing for the bulk properties for the first and/or second component to remain the same. In an example of the ionizing process, RF power is applied at 13.56 Mhz to ionize the gases. In another option, the RF power is about 20 KHz for plasma generation. In yet another option, the RF power is about 10-150 KHz. After generation of plasma, high voltage pulses are applied to the components, and accelerated ions of the material are impinged on the surface of the first and/or second component with high kinetic energy. The incident ions impart energy to substrate atoms via collisions until they are stationary. In an option, at least some of the ions would be buried up to 2000 angstroms under the material surface. In another option, the ions would be buried 300-2000 angstroms under the material surface.

In a further option, after the ion implantation process, or alternatively, independent of the ion implantation process, a coating is deposited on the first portion and/or the second portion of the first and second components, respectively, using plasma polymerization. The coating can be used an interlayer between the components and/or to bond with medical adhesive disposed between the components, as shown in FIG. 2.

In the method for the plasma polymerization process, a material is introduced into the evacuated chamber. The materials include, but are not limited to, methane, ethylene, acetylene, acrylic acid, trimethylsilane. Energy is applied, for example, using a capacitive discharge, for example using 13.56 MHz RF power to generate the plasma, and the plasma polymerization coating can be deposited on the medical device components, such as the cardiac leads components. In another option, the plasma polymerization is deposited in a capacitive discharge in the presence of a magnetic field. In yet another option, a parallel plate DC plasma can be used to plasma polymerize material such as methane, ethylene, or acetylene, with or without the presence of a magnetic field.

In an option, the components are further treated allowing for molecules, such as, but not limited to OH, OOH, or CO₂H molecules, to be formed on the surface of the first component and/or the second component, allowing for bonds therewith, for example, using thermal heating or laser fusion. For instance, a short dose of O₂ plasma is introduced, allowing for the OH bonds. In an option, a few seconds of plasma exposure occurs. In treating the components, OH, OOH, or CO₂H molecules can be released, allowing for a better bond with optional medical adhesive. In another option, the coating of plasma polymerized coating is concentrated on certain portions of the first and/or second component. For example, magnets 196 or a magnetron are used to concentrate the plasma on the components. The magnets allow for less pumping and do not require cooling during the procedure. In another option, the first and/or second component can be moved through concentrated plasma on a ferris wheel type of arrangement, as shown in FIG. 6, or a conveyor system. For the ferris wheel arrangement, an adjustable arm 197 allows for the length to be modified for the component 192 to be treated.

The interlayer between the medical device components allows for a successful bond in a wet environment, for example, within tissue, while maintaining a thin coating. The plasma polymerization process forms highly cross linked structures, allow for improved durability and adhesion under wet and dry conditions.

The first component and the second component are brought together to be coupled together, forming a joint of the medical device. In an option, energy is applied to the first component and/or the second component. For instance, applying energy can include, but is not limited to, thermal heating the first portion of the first component, or applying laser fusion to the first portion of the first component. The energy is applied, and the first component can be bonded with the second component. In a further option, a bonding material, such as medical adhesive, is disposed between the first and second components prior to bonding the components together. Other materials include, but are not limited to, silicone RTV, epoxy, polyurethane adhesive, or UV cure adhesive.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A method for creating an implantable medical device, the method comprising:
evacuating a chamber;
introducing a material into the chamber;
ion implanting a first portion of a first component of the medical device with the material; and
depositing a coating on the first portion of the first component using plasma polymerization.

2. The method of claim 1, further comprising bonding the first component with a second component using the plasma polymerized coating.

3. The method of claim 1, wherein depositing the coating includes depositing the coating on at least one of a metal component or a silicone rubber component.

4. The method of claim 1, further comprising applying energy to the first component.

5. The method of claim 4, wherein applying energy includes thermal heating the first portion of the first component.

6. The electrode of claim 4, wherein applying energy includes applying laser fusion to the first portion of the first component.

7. The method of claim 1, wherein introducing the material into the chamber includes introducing methane into the chamber.

8. The method of claim 1, further comprising ionizing a second portion of a second component with the material, depositing a second coating on the second portion of the second component using plasma polymerization, and bonding the first portion of the first component with the second portion of the second component.

9. The method of claim 1, further comprising treating the first component with at least one of oxygen or hydrogen, and functionalizing the plasma polymerized coating.

10. A method comprising:
disposing a component within a chamber
evacuating the chamber and reducing pressure within the chamber;
introducing a material into the chamber;
generating a plasma polymerized coating using the material; and
depositing the coating on a first portion of a first implantable medical component.

11. The method as recited in claim 10, further comprising bonding the first implantable medical component with a second implantable medical component using the plasma polymerized coating.

12. The method as recited in claim 10, further comprising creating OH molecules on the first implantable medical component.

13. The method as recited in claim 10, further comprising disposing adhesive on the first implantable medical component and bonding the first implantable medical component with a second implantable medical component.

14. The method as recited in claim 10, further comprising concentrating the plasma polymerized coating on portions of the first component.

15. The method of claim 10, further comprising depositing a second plasma polymerized coating on a second portion of a second component, and bonding the first portion of the first component with the second portion of the second component.

16. The method of claim 15, further comprising treating the first implantable medical component and the second implantable medical component with at least one of oxygen or hydrogen, and functionalizing the plasma polymerized coating.

17. The method of claim 10, wherein depositing the coating includes depositing the coating on a joint of the first implantable medical component, and bonding the joint with another component.

18. An implantable medical device comprising:
a first component having a first plasma polymerized portion deposited thereon;
a second component having a second plasma polymerized portion deposited thereon; and
the first plasma polymerized portion is bonded with the second plasma polymerized portion and forming a joint for the medical device.

19. The implantable medical device as recited in claim 18, further comprising medical adhesive disposed between the first plasma polymerized portion and the second polymerized portion.

20. The implantable medical device as recited in claim 18, wherein the first component has a surface treated portion associated with the first plasma polymerized portion.

21. The implantable medical device as recited in claim 20, where the surface treated portion includes an ionized portion.

22. The implantable medical device as recited in claim 18, wherein at least one of the first component or the second component includes an electronically conductive metallic component having a surface enhanced embedded layer.

23. The implantable medical device as recited in claim 18, wherein at least one of the first component or the second component includes at least one of polyurethane or silicone rubber.

24. The implantable medical device as recited in claim 18, wherein the first component includes at least one of an electrode, a terminal, or a lead body.
